(19) United States
(12) Patent Application Publication
(10) Pub. No.: US 2018/0000341 A1
(43) Pub. Date: Jan. 4, 2018

(54) TOMOGRAPHIC IMAGING APPARATUS, TOMOGRAPHIC IMAGING METHOD, IMAGE PROCESSING APPARATUS, IMAGE PROCESSING METHOD, AND PROGRAM

(71) Applicant: CANON KABUSHIKI KAISHA, Tokyo (JP)

(72) Inventors: Nobuhiro Tomatsu, Yokohama-shi (JP); Yoshihiko Iwase, Yokohama-shi (JP); Makoto Sato, Tokyo (JP); Toshiharu Sumiya, Hiratsuka-shi (JP); Makoto Fukuhara, Yokohama-shi (JP); Atsushi Goto, Yokohama-shi (JP); Mitsuro Sugita, North York (CA); Michael Pircher, Vienna (AT); Clemens Vass, Vienna (AT); Christoph Hitzenberger, Vienna (AT)

(21) Appl. No.: 15/540,092

(22) PCT Filed: Dec. 15, 2015
(86) PCT No.: PCT/JP2015/006231

§ 371 Date: Jun. 27, 2017

(30) Foreign Application Priority Data
Jan. 30, 2015 (JP) 2015-017908

(51) Int. Cl. A61B 3/10; A61B 3/00; A61B 3/12; G06T 11/00

(52) U.S. Cl. CPC A61B 3/102; A61B 3/1225; A61B 3/0041; A61B 3/0025; G06T 11/003; G06T 2210/41

(57) ABSTRACT

A alteration caused in a nerve fiber layer could not accurately be displayed. There is provided a tomographic imaging apparatus including a generation means for generating a nerve fiber bundle map, a designation means for designating an arbitrary nerve fiber bundle in the nerve fiber bundle map, and a display control means for causing display means to display a parameter of the designated nerve fiber bundle.

### TECHNICAL FIELD

The present invention relates to a tomographic imaging apparatus, a tomographic imaging method, an image processing apparatus, an image processing method and a program, more particularly, to a tomographic imaging apparatus which can display the characteristic information of the retina located along a nerve fiber bundle of an eye to be examined.

### BACKGROUND ART

Recently, a tomographic imaging (OCT:Optical Coherence Tomography) apparatus (to be referred to as an OCT apparatus hereinafter) using interference caused by low coherence light has been put into practical use. This OCT apparatus can noninvasively acquire a high-resolution tomographic image of an object to be examined, and hence is becoming an indispensable apparatus when obtaining a tomographic image of the fundus of an eye to be examined, especially in the ophthalmology field. In addition, attempts have been made to use this apparatus, outside the ophthalmology field, for example, for tomographic observation of skins and for wall tomography of digestive organs and circulatory organs by forming the apparatus as an endoscope or a catheter.

With regard to an ophthalmologic OCT apparatus, attempts have been made to acquire a functional OCT image by imaging the optical characteristics, movement, and the like of the fundus tissue in addition to a normal OCT image (also called a luminance image) obtained by imaging the shape of the fundus tissue. A polarization OCT apparatus capable of depicting a nerve fiber layer and a retinal layer, in particular, has been developed as one of functional OCT apparatuses, and studies have been made concerning glaucoma, age-related macular degeneration, and the like. In addition, studies have been made to detect a alteration caused in the retinal layer by using a polarization OCT apparatus and determine the progression of a disease and a curative effect.

In diagnosis of glaucoma, perimeter examination is widely practiced. This is a technique of examining how the visual field range of an eye to be examined changes, by using the nature of glaucoma, that is, the occurrence of a visual field defect with the progression of the disease. Recently, there has been disclosed a method of detecting a nerve fiber bundle in which a alteration leading to a visual field defect has occurred, by combining visual field defect information obtained by a perimeter and nerve fiber bundle information with a fundus photograph obtained by a fundus camera (PTL 1).

It is known that with the progression of glaucoma, the thickness of the nerve fiber layer decreases. With regard to diagnosis of glaucoma using an OCT apparatus, studies have been made on a method of dividing a region centered on the optic papilla into a plurality of regions and detecting glaucoma based on the average nerve fiber layer thickness in each region (NPL 1). In addition, a nerve fiber layer has polarization characteristics. Therefore, studies have also been progressed on a technique of grasping a characteristic change before a change in the thickness of a nerve fiber layer and using the grasped change as an index for an early diagnosis (NPL 2).

### CITATION LIST

### Patent Literature

[PTL 1] Japanese Patent No. 3508112

### Non-Patent Literatures

[NPL 1] Arch Ophthalmol. 2004, 122(6):827-837. Felipe A. Medeiros et al. “Comparison of the GDx VCC Scanning Laser Polarimeter, HRT II Confocal Scanning Laser Ophthalmoscope, and Stratus OCT Optical Coherence Tomograph for the Detection of Glaucoma”
[NPL 2] IOVS 2013, 54, 5653, Brad Fortune et al. “Onset and Progression of Peripapillary Retinal Nerve Fiber Layer(RNFL) Retardance Changes Occur Earlier Than RNFL Thickness Changes in Experimental Glaucoma”

### SUMMARY OF INVENTION

### Technical Problem

As described above, glaucoma causes a visual field defect accompanying a alteration in a nerve fiber layer. In addition, a nerve fiber layer is an aggregation of bundles of about 10 μm to 60 μm, called nerve fiber bundles, radially running from the optic papilla. Glaucoma progresses in such a manner that the a characteristic of a nerve fiber layer changes, and the nerve fiber layer decreases in thickness with a change in structure, resulting in changing to a state such as a visual field defect which allows the patient himself/herself to recognize the abnormality. For this reason, when diagnosing and treating glaucoma, it is necessary to recognize as soon as possible a specific portion of a specific nerve fiber bundle, of the nerve fiber bundles constituting the nerve fiber layer, in which an abnormality has occurred. In addition, it is important to grasp the process of this change from a plurality of viewpoints such as layer thickness information and polarization characteristic information.

PTL 1 discloses a method of detecting a nerve fiber bundle in a specific region in which an abnormality leading to a visual field defect has occurred, with respect to the measurement result obtained by a perimeter, by superimposing the distribution pattern of nerve fiber bundles on the fundus image acquired by a fundus camera. For the nerve fiber bundle distribution pattern, the known data is used. According to this method, it is possible to roughly grasp a specific position of a nerve fiber bundle on a fundus image of a patient at which an abnormality has occurred. However, since the nerve fiber bundle distribution pattern to be used does not completely match the actual nerve fiber bundle distribution of the patient, it is difficult to specify an accurate place. In addition, it is not possible to acquire information about the depth position within the fundus and information about polarization characteristics.

NPLs 1 and 2 disclose methods of detecting a place in which an abnormality has occurred, by dividing a region centered on the optic papilla into a plurality of regions, averaging polarization characteristic values such as nerve fiber layer thicknesses or retardation values in each region, and grasping the magnitude or change of each value. However, each method can grasp a change only from the size of each divided region but cannot precisely specify a specific portion in which the change has occurred. In addition, each method cannot present information along an arbitrary nerve fiber bundle.

In consideration of the above problems, the present invention provides a tomographic imaging technology which can present a plurality of pieces of information associated with an extracted nerve fiber bundle.

### Solution to Problem

In order to achieve the above objective, a tomographic imaging apparatus according to the present invention has the following arrangement.

A tomographic imaging apparatus according to the present invention is characterized by comprising generation means for generating a nerve fiber bundle map, designation means for designating an arbitrary nerve fiber bundle in the nerve fiber bundle map, and display control means for causing display means to display a parameter of the designated nerve fiber bundle.

### Advantageous Effects of Invention

According to the present invention, it is possible to provide a tomographic imaging technology which can present a plurality of pieces of information associated with an extracted nerve fiber bundle. Consequently, improvement of the accuracy of diagnosis of glaucoma in the tomographic imaging apparatus can be expected.

Further features of the present invention will become apparent from the following description of exemplary embodiments (with reference to the attached drawings).

### BRIEF DESCRIPTION OF DRAWINGS

FIG. 1 is a schematic view of the overall arrangement of a polarization OCT apparatus according to this embodiment.

FIG. 2A shows an example of an image generated by a signal processing unit 144 according to this embodiment.

FIG. 2B shows an example of an image generated by a signal processing unit 144 according to this embodiment.

FIG. 2C shows an example of an image generated by a signal processing unit 144 according to this embodiment.

FIG. 2D shows an example of an image generated by a signal processing unit 144 according to this embodiment.

FIG. 3 is a flowchart for imaging according to this embodiment.

FIG. 4A explains the extraction of a nerve fiber layer according to this embodiment.

FIG. 4B explains the extraction of a nerve fiber layer according to this embodiment.

FIG. 4C explains the extraction of a nerve fiber layer according to this embodiment.

FIG. 5A explains a nerve fiber bundle tracing method according to this embodiment.

FIG. 5B explains a nerve fiber bundle tracing method according to this embodiment.

FIG. 5C explains a nerve fiber bundle tracing method according to this embodiment.

FIG. 5D explains a nerve fiber bundle tracing method according to this embodiment.

FIG. 6A shows an example of an image to be generated according to this embodiment.

FIG. 6B shows an example of an image to be generated according to this embodiment.

FIG. 7 is a view showing an example of a result output window according to this embodiment.

FIG. 8 is a flowchart for the generation of a nerve fiber bundle map according to this embodiment.

FIG. 9A shows an example of an image to be generated according to this embodiment.

FIG. 9B shows an example of an image to be generated according to this embodiment.

FIG. 9C shows an example of an image to be generated according to this embodiment.

FIG. 9D shows an example of an image to be generated according to this embodiment.

FIG. 10 is a view showing an example of a nerve fiber orientation map according to this embodiment.

FIG. 11 is a view for explaining the generation of a fusion map according to this embodiment.

FIG. 12 is a view showing an example of a fusion map according to this embodiment.

### DESCRIPTION OF EMBODIMENTS

An embodiment of the present invention will be described in detail with reference to the accompanying drawings. This embodiment will particularly exemplify a means for generating a nerve fiber bundle map and tracing a nerve fiber bundle. Note that a nerve fiber bundle map is a map which allows the extraction of information about the running direction of a nerve fiber bundle, and includes, for example, an orientation map and a fusion map. Note that a fusion map will be disclosed in the second embodiment.

### First Embodiment

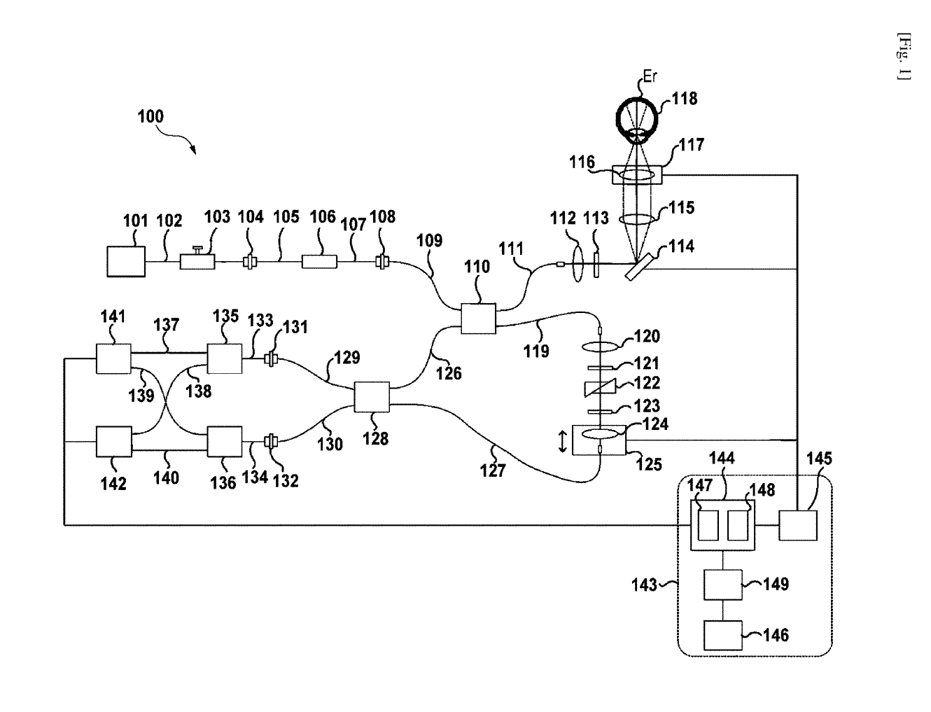

The arrangement of a polarization OCT apparatus according to this embodiment will be described with reference to FIG. 1.

### (Overall Arrangement of Apparatus)

FIG. 1 is a schematic view showing the overall arrangement of a polarization OCT apparatus as an example of a tomographic imaging apparatus according to this embodiment. The embodiment will exemplify a polarization OCT apparatus based on SS (Swept Source)-OCT.

### (Arrangement of Polarization OCT Apparatus 100)

The arrangement of a polarization OCT apparatus 100 will be described.

A light source 101 is a swept source (to be referred to as SS hereinafter) light source, which emits light while performing sweeping with, for example, a sweeping central wavelength of 1,050 nm and a sweeping width of 100 nm.

Light emitted by the light source 101 is guided to a beam splitter 110 via a single mode fiber (to be referred to as SM fiber hereinafter) 102, a polarization controller 103, a connector 104, an SM fiber 105, a polarizer 106, a PM (Polarization Maintaining) fiber (to be referred to as a PM fiber hereinafter) 107, a connector 108, and a PM fiber 109. The guided light is then split into measurement light (to be also referred to as OCT measurement light) and reference light (to be also referred to as reference light corresponding to OCT measurement light). The splitting ratio of the beam splitter 110 is 90 (reference light):10 (measurement light). The polarization controller 103 can change the polarization of light emitted by the light source 101 into a desired polarized state. On the other hand, the polarizer 106 is an optical element having the property of transmitting only a specific linearly polarized light component. Light emitted by the light source 101 contains, as dominant light, light having a high polarization degree and a specific polarization direction, but also contains light having no specific polarization direction, which is called randomly polarized light components. It is known that such randomly polarized light components degrade the image quality of a polarization OCT image. Therefore, randomly polarized light components are cut by the polarizer. Note that since only light in a specific linearly polarized state can pass through the polarizer 106, the polarization controller 103 adjusts a polarized state so as to make a desired amount of light enter an eye 118 to be examined.

The split measurement light exits via a PM fiber 111 and is collimated by a collimator 112. The collimated measurement light is transmitted through a ¼ wavelength plate 113 and then enters the eye 118 via a galvano scanner 114 which scans measurement light on a fundus Er of the eye 118, a scan lens 115, and a focus lens 116. In this case, the galvano scanner 114 is drawn as a single mirror, but is actually constituted by two galvano scanners to raster-scan the fundus Er of the eye 118. In addition, the focus lens 116 is fixed on a stage 117, and can perform focus adjustment by moving in the optical axis direction. A drive control unit 145 controls the galvano scanner 114 and the stage 117 to scan measurement light in a desired range (also called a tomographic image acquisition range, tomographic image acquisition position, or measurement light irradiation position) on the fundus Er of the eye 118. The ¼ wavelength plate 113 is an optical element having the property of delaying the phase between the optical axis of the ¼ wavelength plate and an axis perpendicular to the optical axis by ¼ wavelength. In this embodiment, the optical axis of the ¼ wavelength plate 113 is rotated through 45° about the optical axis as a rotation axis with respect to the linearly polarizing direction of measurement light exiting from the PM fiber 111 to convert light entering the eye 118 into circularly polarized light. Although not described in detail in this embodiment, it is preferable to provide a tracking function of detecting the movement of the fundus Er and scanning the mirror of the galvano scanner 114 following the movement of the fundus Er. It is possible to perform a tracking method by using a general technique and perform the method in real time or in a postprocessing step. For example, a method using an SLO (Scanning Laser Ophthalmoscope) is available. This method is configured to acquire a two-dimensional image of the fundus Er in a plane perpendicular to the optical axis over time by using an SLO and extract a feature portion such as a blood vessel branch or the like in the image. It is possible to perform real-time tracking by calculating, as the movement amount of the fundus Er, how a feature portion in an acquired two-dimensional image has moved, and feeding back the calculated movement amount to the galvano scanner 114.

The focus lens 116 mounted on the stage 117 makes measurement light enter the eye 118 so as to focus the light on the fundus Er. The measurement light irradiating the fundus Er is reflected/scattered by each retinal layer to return to the beam splitter 110 along the above optical path. Return light of the measurement light entering the beam splitter 110 enters a beam splitter 128 via a PM fiber 126.

The reference light split by the beam splitter 110 exits via a PM fiber 119 and is collimated by a collimator 120. The reference light enters a PM fiber 127 via a ½ wavelength plate 121, a dispersion-compensating glass 122, an ND filter 123, and a collimator 124. The collimator 124 and one end of the PM fiber 127 are fixed on a coherence gate stage 125, which is controlled by the drive control unit 145 to drive in the optical axis direction in accordance with, for example, the difference in eye axial length between subjects. The ½ wavelength plate 121 is an optical element having the property of delaying the phase between the optical axis of the ½ wavelength plate and an axis perpendicular to the optical axis by ½ wavelength. In this embodiment, the linearly polarized light of reference light exiting from the PM fiber 119 is adjusted into a polarized state in which the long axis of the linearly polarized light tilts by 45° in the PM fiber 127. Note that the embodiment is configured to change the optical path length of reference light, but is only required to change the optical path length difference between the optical path of measurement light and the optical path of reference light.

Reference light passing through the PM fiber 127 enters the beam splitter 128. At the beam splitter 128, the return light of the reference light and the returning measurement light are multiplexed into interference light, which is then split into two light components. The split interference light includes interference light components having inverted phases (to be expressed as a positive component and a negative component, respectively). The positive component of the split interference light enters a polarization beam splitter 135 via a PM fiber 129, a connector 131, and a PM fiber 133. On the other hand, the negative polarized light component of the interference light enters a polarization beam splitter 136 via a PM fiber 130, a connector 132, and a PM fiber 134.

The polarization beam splitters 135 and 136 split interference light, in accordance with two orthogonal polarization axes, into two light components, namely a vertically polarized light component (to be referred to as a V polarized light component hereafter) and a horizontally polarized light component (to be referred to as an H polarized light component hereinafter). The positive interference light entering the polarization beam splitter 135 is split into two interference light components, namely a positive V polarized light component and a positive H polarized light component, respectively, at the polarization beam splitter 135. The split positive V polarized light component enters a detector 141 via a PM fiber 137. The positive H polarized light component enters a detector 142 via a PM fiber 138. On the other hand, the negative interference light entering the polarization beam splitter 136 is split into a negative V polarized light component and a negative H polarized light component at the polarization beam splitter 136. The negative V polarized light component enters the detector 141 via a PM fiber 139. The negative H polarized light component enters the detector 142 via a PM fiber 140.

Both the detectors 141 and 142 are differential detectors. Upon receiving two interference signals whose phases are inverted by 180° from each other, each detector removes DC components and outputs only interference components.

The V polarized light component of the interference signal detected by the detector 141 and the H polarized light component of the interference signal detected by the detector 142 are output as electrical signals respectively corresponding to the light intensities, which are input to a signal processing unit 144 as an example of a tomographic image generation unit.

(Control Unit 143)

The control unit 143 for controlling the overall apparatus will be described.

The control unit 143 is constituted by the signal processing unit 144, the drive control unit 145, a display unit 146, and a display control unit 149. The signal processing unit 144 further includes a fundus image generation unit 147 and a map generation unit 148. The fundus image generation unit 147 has a function of generating a luminance image and a polarization characteristic image from the electrical signal sent from the signal processing unit 144. The map generation unit 148 has a function of generating a nerve fiber bundle map and a nerve fiber bundle trace map.

The drive control unit 145 controls the respective units in the manner described above. The signal processing unit 144 generates an image, analyzes the generated image, and generates visualization information as an analysis result based on the signals output from the detectors 141 and 142.

The image and the analysis result generated by the signal processing unit 144 are sent to the display control unit 149. The display control unit 149 causes the display unit 146 to display the image and the analysis result on the display screen. In this case, the display unit 146 is a display such as a liquid crystal display. Note that the image data generated by the signal processing unit 144 may be transmitted to the display unit 146 wiredly or wirelessly after being sent to the display control unit 149. In addition, in this embodiment, the display unit 146 and the like are included in the control unit 143, but the present invention is not limited to this, and they may be provided separately from the control unit 143. For example, the display unit 146 and the like may be provided as a tablet which is an example of a device which can be carried by the user. In this case, the display unit is preferably equipped with a touch panel function and configured to allow the user to perform operations to, for example, move the display position of an image, enlarge/reduce the image, and change the image to be displayed on the touch panel.

### (Image Processing)

Image generation in the signal processing unit 144 will be described next. The signal processing unit 144 generates tomographic images respectively corresponding to an H polarized light component and a V polarized light component, which are two tomographic images based on the respective polarized light components, by performing general reconstruction processing in the fundus image generation unit 147 with respect to the interference signals output from the detectors 141 and 142.

First of all, the fundus image generation unit 147 removes fixed pattern noise from an interference signal. Fixed pattern noise removal is performed by extracting fixed pattern noise by averaging a plurality of detected A-scan signals, and then subtracting the noise from an input interference signal. The fundus image generation unit 147 then performs desired window function processing to optimize a depth resolution and a dynamic range which have a tradeoff relationship when performing Fourier transform in a finite interval. Thereafter, the fundus image generation unit 147 generates a tomographic signal by performing FFT processing.

Two tomographic images are generated by performing the above processing on interference signals of two polarized light components. A luminance image and a polarization characteristic image are generated based on these tomographic signals and tomographic images. A polarization characteristic image is obtained by imaging the polarization characteristics of an eye to be examined. Such images include, for example, an image based on retardation information, an image based on orientation information, and an image based on birefringence information. The image based on retardation information includes, for example, a retardation image and a retardation map which are described later. The image based on orientation information includes, for example, an orientation map which is described later. The image based on luminance includes, for example, a luminance image map, a nerve fiber layer thickness map, and a fiber bundle orientation map, which are described later.

(Generation of Luminance Image)

The fundus image generation unit 147 generates a luminance image from the two tomographic signals described above. The luminance image is basically the same as a tomographic image in conventional OCT, and a pixel value r is calculated from a tomographic signal A_{H} of the H polarized light component and a tomographic signal A_{V} of the V polarized light component obtained from the detectors 141 and 142 according to equation (1).

r=\sqrt{A_{H}²+A_{V}²} (1)

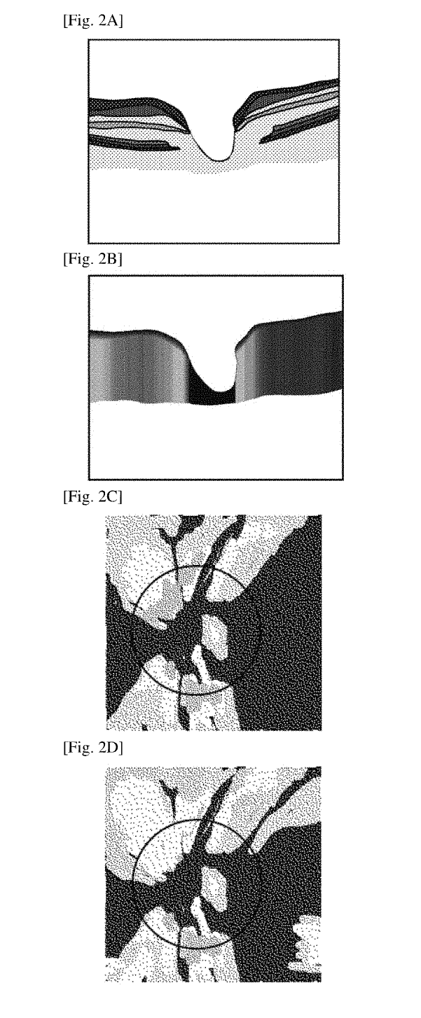

In FIG. 2A, FIG. 2A shows an example of a luminance image of the optic papilla.

In addition, the galvano scanner 114 performs raster scanning to arrange B-scan images of the fundus Er of the eye 118 in the sub-scanning direction, thereby generating the volume data of the luminance image.

(Generation of Retardation Image)

The fundus image generation unit 147 generates a retardation image from tomographic images of orthogonal polarized light components.

A value δ of each pixel of a retardation image numerically expresses the phase difference between a vertically polarized light component and a horizontally polarized light component at the position of each pixel of a tomographic image, and is calculated from the tomographic signals A_{H} and A_{V} according to equation (2).

$$δ = arctan [ \frac{{A}_{V}}{{A}_{H}} ] \qquad ( 2 )$$

In FIG. 2B, FIG. 2B shows an example of a retardation image (also called a tomographic image indicating the phase difference between polarized light) of the optic papilla generated in this manner, which can obtained by calculating equation (2) with respect to each B-scan image. In FIG. 2B, a portion of a tomographic image in which a phase difference occurs is displayed in color, with a place with dark shading indicating a small phase difference, and a place with light shading indicating a large phase difference. Therefore, generating a retardation image makes it possible to grasp a layer having birefringence.

(Generation of Retardation Map)

The fundus image generation unit 147 generates a retardation map from a retardation image obtained with respect to a plurality of B-scan images.

First of all, the signal processing unit 144 detects an RPE (Retinal Pigment Epithelium) in each B-scan image. The RPE has the property of scrambling polarization. For this reason, the distribution of retardations in each A-scan is checked in a range from the ILM (Inner Limiting Membrane), along the depth direction, excluding the RPE, and the maximum value in the distribution is set as a representative value of the retardations in the A-scan.

The fundus image generation unit 147 generates a retardation map by performing the above processing on all the retardation images.

In FIG. 2C, FIG. 2C shows an example of a retardation map of the optic papilla. Referring to FIG. 2C, a place with dark shading indicates a small phase difference, and a place with light shading indicates a large phase difference. Around the optic papilla, the RNFL (Retinal Nerve Fiber Layer) is a layer having birefringence, and the retardation map indicates phase differences caused by the birefringence of the RNFL and the thickness of the RNFL. For this reason, a portion in which the RNFL is thick has a large phase difference, and a portion in which the RNFL is thin has a small phase difference. It is therefore possible to grasp the thickness of the RNFL of the overall fundus from the retardation map. This makes it possible to use the map for the diagnosis of glaucoma.

(Generation of Birefringence Map)

The fundus image generation unit 147 linearly approximates the value of the retardation 8 in each A-scan image of previously generated retardation images within the range from the ILM to the RNFL (Retinal Nerve Fiber Layer), and designates the slope of the approximation as a birefringence at a position on the retina corresponding to the A-scan position. A map representing birefringence is generated by performing this processing on all the acquired retardation images.

In FIG. 2D, FIG. 2D shows an example of the birefringence map of the optic papilla region. The birefringence map is obtained by directly mapping birefringence values, and hence can depict a change in the fiber structure of the RNFL as a change in birefringence even if the thickness of the RNFL does not change.

(Processing Operation)

The processing operation in this polarization OCT apparatus will be described next.

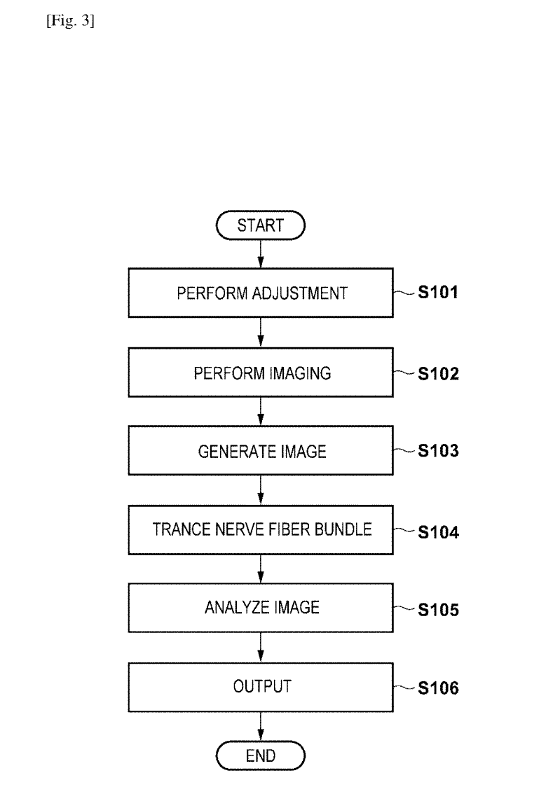

FIG. 3 is a flowchart showing the processing operation in this polarization OCT apparatus.

(Adjustment)

First of all, in step S101, while an eye to be examined is located at this apparatus, alignment is performed between the apparatus and the eye. Note that since alignment in the X, Y, and Z directions for a working distance or the like, focus adjustment, coherence gate adjustment, and the like are the same as those in general OCT, a description of them will be omitted.

(Imaging to Image Generation)

In steps S102 and S103, the light source 101 emits light to generate measurement light and reference light. The detectors 141 and 142 receive interference light between the reference light and the return light of the measurement light reflected or scattered by the fundus Er of the eye 118. The signal processing unit 144 then generates each image in the manner described above.

(Tracing of Nerve Fiber Bundle)

The processing performed in step S104 by the map generation unit 148 which is one function of the signal processing unit 144 will be described below.

(Extraction of Nerve Fiber Layer)

The map generation unit 148 extracts a nerve fiber layer by performing segmentation using the luminance image generated in step S103.

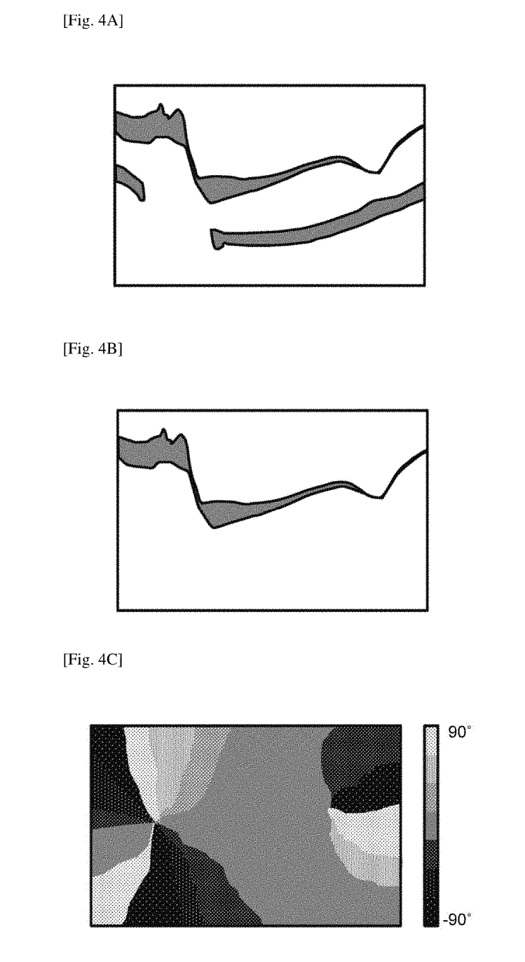

First of all, the map generation unit 148 binarizes the generated luminance image. Each pixel equal to or more than a threshold set in advance by the operator is set to 1, and each pixel equal to or less than the threshold is set to 0. The operator can arbitrarily set a threshold in accordance with the image quality of an image. In FIG. 4A, FIG. 4A shows the binarized image.

The data of a region corresponding to a nerve fiber layer is extracted from the generated binarized image. The generated binarized image has luminance values in a wide spatial range including the nerve fiber layer, and there are pixels having luminance values on a retinal layer other than the nerve fiber layer. The nerve fiber layer exists at the top of the retinal layer, that is, on the vitreous body side, and hence pixels existing on the vitreous body side of the binarized image are selectively extracted.

Attention is paid to a group of pixels, on the generated binarized image, which are arranged in the A-scan direction, that is, the depth direction of the image. The values of pixels are sequentially checked from the vitreous body side to extract a pixel group continuously having luminance values in the interval from the first pixel having the value of 1 to the first pixel whose pixel value becomes 0. This processing is sequentially executed on all the A-scans in the B-scan image. This makes it possible to selectively extract a nerve fiber layer. In FIG. 4B, FIG. 4B shows an image obtained by extracting the nerve fiber layer.

(Generation of Orientation Map)

The map generation unit 148 generates an orientation map. The orientation map two-dimensionally displays the running direction of nerve fiber bundles radially extending from the optic papilla. That is, it is possible to visualize the running direction of nerve fiber bundles by generating an orientation map. Therefore, an orientation map is generated by visualizing the distribution of the running direction of nerve fiber bundles. An orientation is a parameter indicating the direction of anisotropy when it exists in a given structure. In the case of a nerve fiber layer, nerve fiber bundles radially expand from the optic papilla as the center. In addition, a nerve fiber bundle is a tissue having anisotropy, and differs in refractive index in the running direction and a direction perpendicular to the running direction. When light enters the nerve fiber bundle, therefore, a component polarized along the running direction of the nerve fiber bundle delays with respect to a component polarized perpendicular to the running direction. At this time, a polarization direction corresponding to delayed light, that is, the running direction of the nerve fiber bundle, becomes a delayed phase axis, and a direction perpendicular to the running direction becomes an advanced phase axis. An orientation is a parameter indicating the direction of a delayed phase axis, in particular, in a structure having anisotropy. In the case of a nerve fiber layer, an orientation is a parameter indicating the direction of the nerve fiber bundle. For this reason, calculating an orientation for each pixel of an acquired OCT image can obtain the running direction information of the nerve fiber bundle at each pixel. An orientation can be obtained by using a phase difference ΔΦ between each tomographic signal A_{H} and the corresponding tomographic signal A_{V} according to equation (3).

$$θ = \frac{π - Δφ}{2} \qquad ( 3 )$$

An orientation map is generated by performing the above processing on all acquired images. In FIG. 4C, FIG. 4C shows an example of an orientation map.

(Detection of Optic Papilla and Macula)

The map generation unit 148 traces nerve fiber bundles.

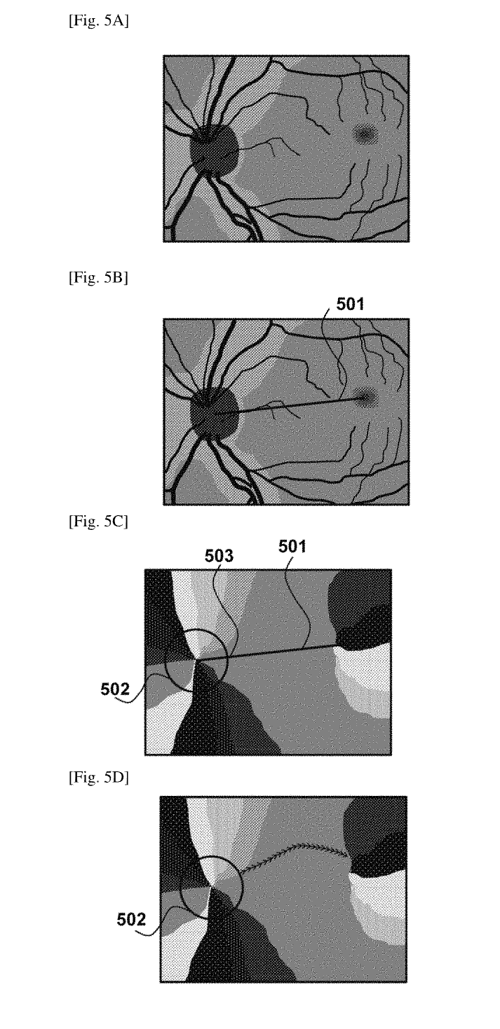

First of all, the map generation unit 148 generates a luminance image map based on the luminance image generated in step S103. The luminance image map is obtained by averaging luminance values in the A-scan direction, that is, the depth direction, with respect to the generated luminance image, and two-dimensionally arranging the resultant values. In FIG. 5A, FIG. 5A shows the luminance image map.

An optic papilla and a macula are detected from the generated luminance image map. In general, there are few high reflective layers in regions of the optic papilla and macula on an OCT tomographic image. For this reason, on the luminance image map, regions of the optic papilla and macula are lower in luminance than other regions. It is therefore possible to detect the optic papilla and the macula by extracting regions with low luminance values. When detecting the optic papilla and the macula, a threshold is provided for the luminance values of the luminance image map, and regions with luminance values equal to or less than the threshold are extracted. The barycenters of the extracted regions are calculated, and the obtained coordinates are set as the centers of the optic papilla and macula, respectively. Note that an examiner may arbitrarily set a threshold so as to selectively detect the optic papilla and the macula. In addition, a method of detecting the optic papilla and the macula is not limited to the method described above. It is possible to use any generally practiced detection methods. For example, shape information combined with the luminance image could be utilized for optic papilla and macula. In addition, although this embodiment has exemplified the method of automatically detecting above regions using the signal processing unit, the examiner may manually extract the above regions from any fundus images of the patient.

(Setting of Reference Coordinates)

Upon detecting the optic papilla and the macula, the map generation unit 148 sets reference coordinates 501 on the orientation image generated as shown in FIG. 5B. The map generation unit 148 sets a coordinate system in which the direction of a straight line connecting the center the detected optic papilla to the center of the detected macula is set to 0°, and clockwise angles around the center of the optic papilla as an axis become positive angles. Although in this embodiment, the straight line connecting the optic papilla and the macula is set as reference coordinates, the present invention is not limited to this. It is possible to set any coordinate system as needed. In addition, although in the embodiment, the coordinate system is set such that clockwise angles around the center of the optic papilla become positive angles, the present invention is not limited to this, and the examiner may arbitrarily set a coordinate system.

(Tracing of Nerve Fiber Bundles)

Upon deciding coordinates, the map generation unit 148 traces nerve fiber bundles. The map generation unit 148 starts tracing from a position at an arbitrary distance from the central portion of the optic papilla. This is because, since the optic papilla is recessed, the nerve fiber layer abruptly declines, an orientation value at the optic papilla may be inaccurate. For this reason, in this embodiment, as indicated by FIG. 5C, a circle 502 with a diameter of 1.8 mm is drawn centered on the optic papilla, and the map generation unit 148 starts tracing from a position on the circumference of the circle. In addition, in the embodiment, the reference position for tracing is set to an intersection point 503 between the reference coordinate axis connecting the center of the optic papilla and the center of the macula and the circle 502 around the optic papilla, and tracing is performed clockwise, from 0° to 359°, on the circumference from the reference position. When tracing comes to 360°, the tracing position becomes the same position as that of 0°. This circumference is divided in increments of 1° to set a total tracing count of 360, and tracing is performed from each position. Note that the tracing start position and the number of nerve fiber bundles to be traced are not limited to them. The operator may decide a start position or the number of nerve fiber bundles to be traced by using an arbitrary means.

The map generation unit 148 performs tracing from each tracing start position on the circumference in accordance with an orientation value. First of all, the map generation unit 148 decides a tracing direction based on the orientation value at the pixel at the start position of 0°. At this time, the orientation value contains only direction information but contains no distance information. For this reason, in this embodiment, the distance by which tracing is to be performed in accordance with the orientation value at one pixel is set to 35 μm. This makes it possible to decide the position of a pixel at which the next orientation value is to be extracted. That is, the map generation unit 148 decides the direction of a nerve fiber bundle at the pixel at the start position of 0° based on the orientation value at the pixel, and makes the tracing position advance by a predetermined distance, 35 μm in this embodiment. At the next pixel in the forward direction, the direction of the nerve fiber bundle has changed. Along with this change, the orientation value at the pixel has also changed. For this reason, the map generation unit 148 decides a tracing direction again from the orientation value at the corresponding pixel, and then makes the tracing position advance by 35 μm. In this manner, the map generation unit 148 repeats the process of deciding a direction from the orientation value at the next pixel in the forward direction in the same manner and making the tracing position advance by a predetermined distance. The above method makes it possible to trace first a nerve fiber bundle passing through the position of 0°. Performing tracing from other start positions 1° to 359° in the same manner can trace nerve fiber bundles passing through the circumference centered on the optic papilla (FIG. 5D). Although in this embodiment, the distance by which tracing is performed with respect to one pixel is set to 35 μm, the present invention is not limited to this. It is possible to arbitrarily set a distance in accordance with the resolution and accuracy required by the operator as long as the distance is at least equal to or more than the pixel size of an orientation map.

(Image Analysis)

The processing performed in step S105 by the map generation unit 148 will be described below.

(Generation of Nerve Fiber Bundle Trace Map)

The map generation unit 148 generates a nerve fiber bundle trace map after tracing all the 360 nerve fiber bundles. A nerve fiber bundle trace map expresses the characteristics of nerve fiber bundles, for example, the birefringence, retardation, and thickness, in colors, with the abscissa representing all the traced nerve fiber bundles, and the ordinate representing the lengths of the nerve fiber bundles.

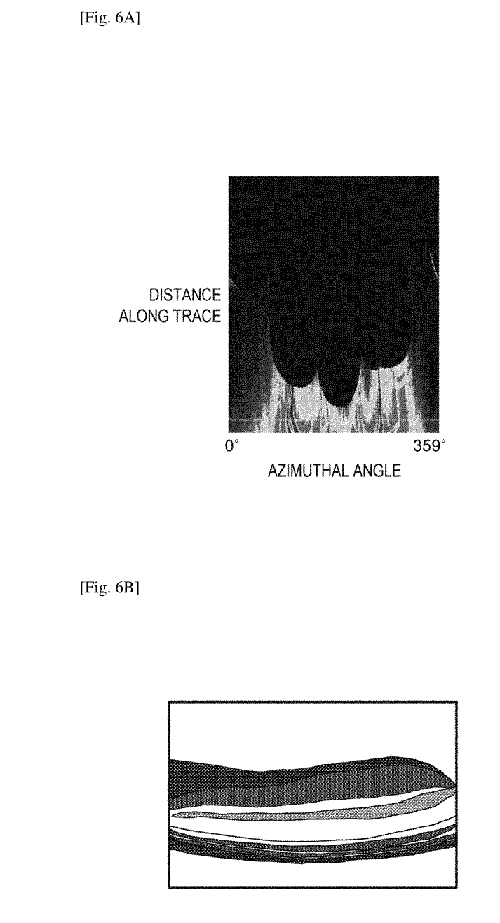

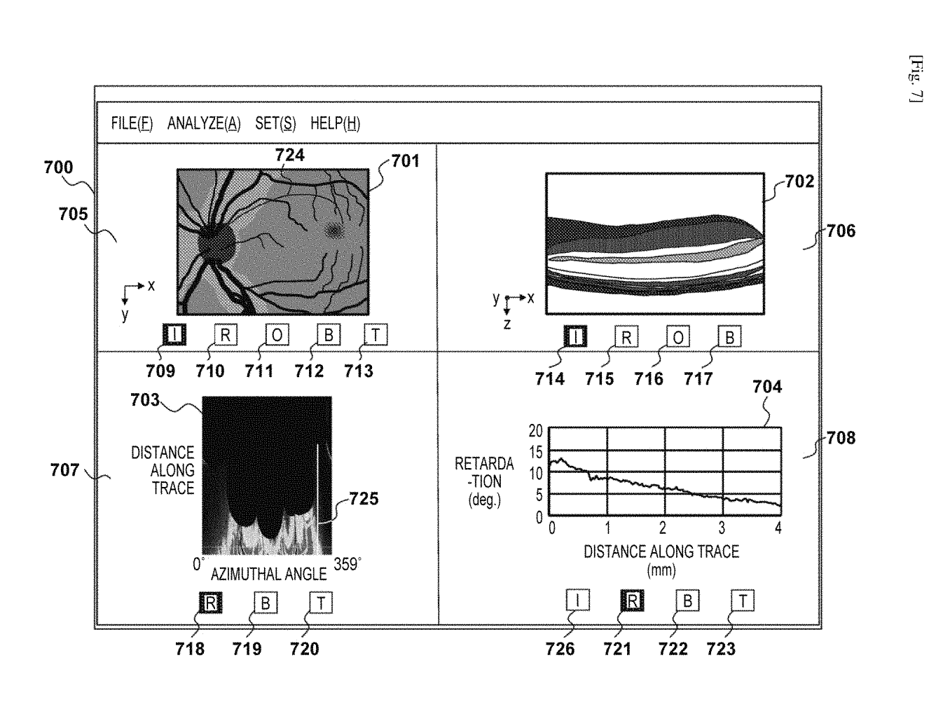

The map generation unit 148 obtains coordinate values corresponding to the respective traced nerve fiber bundles, and extracts retardation values, birefringence values, and thickness values at the respective coordinates from the retardation map, the birefringence map, and the thickness map. The map generation unit 148 then displays the retardation values, the birefringence values, and the thickness values as color gradations on one image, with the abscissa representing the traced nerve fiber bundles, the ordinate representing the lengths of the respective nerve fiber bundles. Generating such a map makes it possible to easily check a specific state of each nerve fiber bundle extending from the optic papilla at a specific angle. In FIG. 6A, FIG. 6A shows a nerve fiber bundle trace map concerning retardation.

Although in this embodiment, a nerve fiber bundle trace map is displayed with the abscissa representing the traced nerve fiber bundles and the ordinate representing the lengths of the respective nerve fiber bundles, the present invention is not limited to this. It is possible to arbitrarily set ordinate and abscissa as necessary.

(Generation of Luminance Image along Nerve Fiber Bundle)

The signal processing unit 144 generates a luminance image along a traced nerve fiber bundle. The signal processing unit 144 extracts A-scan data corresponding to a place at the coordinate values of each traced nerve fiber bundle from the volume data of the luminance image generated in the above manner, and reconstructs the extracted data as a luminance tomographic image for each nerve fiber bundle. In FIG. 6B, FIG. 6B shows a luminance image along a nerve fiber bundle.

(Image Output)

After information along a nerve fiber bundle is acquired in step S105, the signal processing unit 144 sends output information to the display control unit 149 in step S106. The display control unit 149 further sends the information to the display unit 146. The display unit 146 displays the received respective types of information.

(Image Display Window)

FIG. 7 shows an display example of the display unit 146 in this embodiment. Referring to FIG. 7, a window 700 displayed on the display unit 146 includes display areas 705, 706, 707, and 708.

A fundus image 701 is displayed in the display area 705 (also called the first display area). Buttons 709 to 713 (examples of selection portions) for selecting the type of image to be displayed are also displayed in the display area 705 to make it possible to display images based on polarization signals as well as luminance signals. For example, it is possible to display various types of fundus images in a plane perpendicular to the optical axis of measurement light, for example, a retardation map, orientation map, birefringence map, and nerve fiber layer thickness map. Note that the type of image may be selected from a menu instead of the buttons 709 to 713. FIG. 7 shows an example of displaying a luminance image map, with the button 709 being selected. The remaining buttons 710 to 713 and corresponding displays will be described. When the operator presses the button 710, a retardation map is displayed. When the operator presses the button 711, an orientation map is displayed. When the operator presses the button 712, a birefringence map is displayed. When the operator presses the button 713, a nerve fiber layer thickness map is displayed. Note that it is preferable to display a luminance image map, retardation map, orientation map, birefringence map, nerve fiber layer thickness map, and the like while superimposing display patterns indicating the respective types of images (for example, the word “Intensity”, the word “Retardation”, the word “Orientation”, the word “Birefringence”, and the word “Thickness”) on the respective images. This can prevent the operator from falsely recognizing each image. Obviously, such a display pattern may be displayed on, for example, the upper or lateral side of an image, instead of being superimposed on the image, so as to be displayed in correspondence with the image.

A nerve fiber bundle trace map 703 of retardation is displayed in the display area 707 (also called the second display area). Buttons 718 to 720 (examples of selection portions) are displayed in the display area 707 to make it possible to display not only a retardation map but also nerve fiber bundle trace maps of birefringence, nerve fiber layer thickness, and the like. In this embodiment, when the operator presses the button 718, a nerve fiber bundle trace map of retardation is displayed. When the operator presses the button 719, a nerve fiber bundle trace map of birefringence. When the operator presses the button 720, a nerve fiber bundle trace map of nerve fiber layer thicknesses is displayed. Note that it is preferable to display various types of nerve fiber bundle trace maps while superimposing display patterns indicating the respective types of images (for example, the word “Retardation”, the word “Birefringence”, and the word “Thickness”) on the respective images. Obviously, such a display pattern may be displayed on, for example, the upper or lateral side of an image, instead of being superimposed on the image, so as to be displayed in correspondence with the image. This can prevent the operator from falsely recognizing each image.

A fundus tomographic image 702 along a nerve fiber bundle is displayed in the display area 706 (also called the third display area). Buttons 714 to 717 (examples of selection portions) are displayed in the display area 706 to display a tomographic image using a polarization image as well as a luminance image. For example, retardation image, orientation image, birefringence image, or the like along the nerve fiber bundle can be displayed in the display area 706. In this embodiment, when the operator presses the button 714, a luminance tomographic image along a selected nerve fiber bundle is displayed. When the operator presses the button 715, a retardation image along a selected nerve fiber bundle is displayed. When the operator presses the button 716, an orientation image along a selected nerve fiber bundle is displayed. When the operator presses the button 717, a birefringence image along a selected nerve fiber bundle is displayed. Note that it is preferable to display the fundus tomographic image 702 along a nerve fiber bundle while superimposing a corresponding one of display patterns indicating the respective types of images (for example, the word “Intensity”, the word “Retardation”, the word “Orientation”, and the word “Birefringence”) on the image. This can prevent the operator from falsely recognizing each image. Obviously, such a display pattern may be displayed on, for example, the upper or lateral side of an image, instead of being superimposed on the image, so as to be displayed in correspondence with the image.

The characteristic information 704 along a nerve fiber bundle is displayed in the display area 708 (also called the fourth display area). Buttons 721 to 723 (examples of selection portions) are displayed in the display area 708 to allow the operator to arbitrarily change the information to be displayed. In this embodiment, when the operator presses the button 721, retardation information at a position along a selected nerve fiber bundle can be displayed in the form of a graph. When the operator presses the button 722, birefringence information at a position along a selected nerve fiber bundle can be displayed in the form of a graph. When the operator presses the button 723, nerve fiber layer thickness information at a position along the running direction of a selected nerve fiber bundle can be displayed in the form of a graph. When the operator presses a button 726, luminance information at a position along a selected nerve fiber bundle can be displayed in the form of a graph. Note that it is preferable to display the respective types of nerve fiber bundle trace maps while superimposing display patterns indicating the respective types of images (for example, the word “Retardation”, the word “Birefringence”, the word “Thickness”, and the word “Intensity”) on the respective images. Obviously, such a display pattern may be displayed on, for example, the upper or lateral side of an image, instead of being superimposed, so as to be displayed in correspondence with the image. This can prevent the operator from falsely recognizing each image.

(Operation Procedure)

First of all, the operator designates an arbitrary place in the fundus image 701 displayed in the display area 705 of the window 700. When performing this operation, it is possible to use any technique capable of designating an arbitrary place, for example, a pointer such as a mouse. In addition, this embodiment has exemplified the technique of designating an arbitrary place. However, for example, the embodiment may use a scheme of displaying the respective nerve fiber bundles in the form of a list and letting the operator select an arbitrary nerve fiber bundle from the list. In the embodiment, the above operation is performed while the button 709 is pressed and a luminance image is displayed. Obviously, however, it is possible to perform the operation by using another tomographic image displayed by pressing a corresponding one of the buttons 710 to 713. When an arbitrary place is designated in the fundus image 701, the display unit 146 sends coordinate information to the signal processing unit 144, and the signal processing unit 144 extracts the information of a nerve fiber bundle 724 passing through the selected place based on the coordinate information. The extracted information is sent to the display unit 146 and is displayed in the display areas 706 and 708. In addition, at this time, the nerve fiber bundle 724 extracted in the fundus image 701 is highlighted to allow the operator to recognize the running direction of the nerve fiber bundle in the fundus image 701. Furthermore, at the same time, it is preferable to highlight a portion, in the nerve fiber bundle trace map 703, which indicates a nerve fiber bundle 725 corresponding to the nerve fiber bundle 724 in the display area 707. When the tomographic image 702 along the nerve fiber bundle and the characteristic information 704 along the nerve fiber bundle are displayed, the operator can change the images by arbitrarily pressing the buttons 714 to 717 and the buttons 721 to 723 and 726.

It is also possible to display the tomographic image 702 along the nerve fiber bundle and the characteristic information 704 along the nerve fiber bundle by designating an arbitrary point on the nerve fiber bundle trace map 703. In this case, when an arbitrary place is designated on the nerve fiber bundle trace map 703, the corresponding coordinate information is sent to the signal processing unit 144, and information about the nerve fiber bundle 725 passing through the coordinates is extracted. Although in this embodiment, the nerve fiber bundle trace map of retardation is used by pressing the button 718, the present invention is not limited to this. It is possible to perform the above operation based on other types of nerve fiber bundle trace maps by pressing the buttons 719 and 720. Extracted information is sent to the display unit 146 and is displayed in the display areas 706 and 708. In addition, at this time, the extracted nerve fiber bundle 725 in the nerve fiber bundle trace map 703 is highlighted to allow the operator to recognize the position of the nerve fiber bundle in the nerve fiber bundle trace map 703. In addition, at the same time, it is preferable to highlight a portion, in the fundus image 701, which indicates the nerve fiber bundle 724 corresponding to the nerve fiber bundle 725 in the nerve fiber bundle trace map 703 in the display area 705.

Using the polarization OCT apparatus described above makes it possible to display information along nerve fiber bundle. In addition, it is possible to individually diagnose the state of each nerve fiber bundle by using the above polarization OCT apparatus. This leads to early detection of a nerve fiber bundle defect. In addition, it is possible to grasp the relationship between a nerve fiber bundle defect and a visual field defect. In addition, grasping the overall state of nerve fiber bundles will lead to early diagnosis of glaucoma. Although this embodiment has exemplified imaging and displaying in SS-OCT, the present invention is not limited to this. This method can be applied to any apparatuses which can obtain polarization OCT images. In addition, the embodiment has exemplified the display operation using a retardation image, orientation image, birefringence image, and luminance image and the displaying of characteristic information. However, the present invention is not limited to this. This method can use any kind of information about tissues constituting the fundus, such as the retina and choroid membrane. In addition, although in the embodiment, a nerve fiber bundle is detected by using orientation, the present invention is not limited to this. It is possible to use any method capable of detecting a nerve fiber bundle. In such a case, this method can be used by aligning a polarization OCT image with a luminance image. Furthermore, the embodiment includes the fundus image generation unit 147 and the map generation unit 148 each as one function of the signal processing unit 144. If, however, the signal processing unit 144 does not have such distinctive functions and processes, the signal processing unit 144 may be configured to generate fundus images and maps by itself.

### Second Embodiment

This embodiment will exemplify a method of generating a fusion map by using both a fiber bundle orientation map and an orientation map.

(Overall Arrangement of Apparatus and Signal Acquisition)

An apparatus arrangement and signal acquisition are the same as those of the polarization OCT apparatus 100 described in the first embodiment, and hence a description of them will be omitted.

(Image Processing)

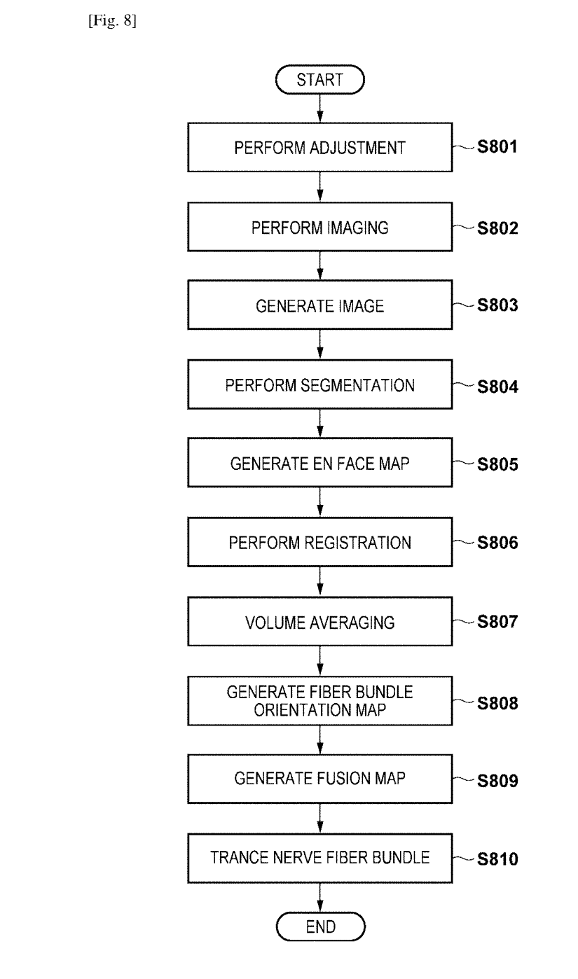

A procedure for generating a nerve fiber bundle map by using a fiber bundle orientation map and an orientation map will be described with reference to FIG. 8.

(Adjustment to Imaging)

First of all, in step S801, while an eye to be examined is located at this apparatus, alignment is performed between the apparatus and the eye. In step S802, imaging is performed. In imaging, a scanner 114 is raster-scanned to acquire volume data. In addition, in order to reduce instability and noise components of obtained data, it is preferable to perform averaging processing using a plurality of images in the subsequent processing. For this purpose, in this embodiment, five images are acquired. The contents of adjustment and imaging are the same as those described in the first embodiment, and hence a description of the contents will be omitted. Note that the number of images to be acquired is not limited to five, and the examiner can arbitrarily decide the number of images. In addition, although in the embodiment, a plurality of images are obtained, it is possible to perform imaging for only one image and omit the subsequent averaging processing.

(Image Generation)

In step S803, a fundus image generation unit 147 as one function of a signal processing unit 144 generates a luminance image from an acquired signal. A method of generating a luminance image is the same as that described in the first embodiment, and hence a description of the method will be omitted.

(Segmentation)

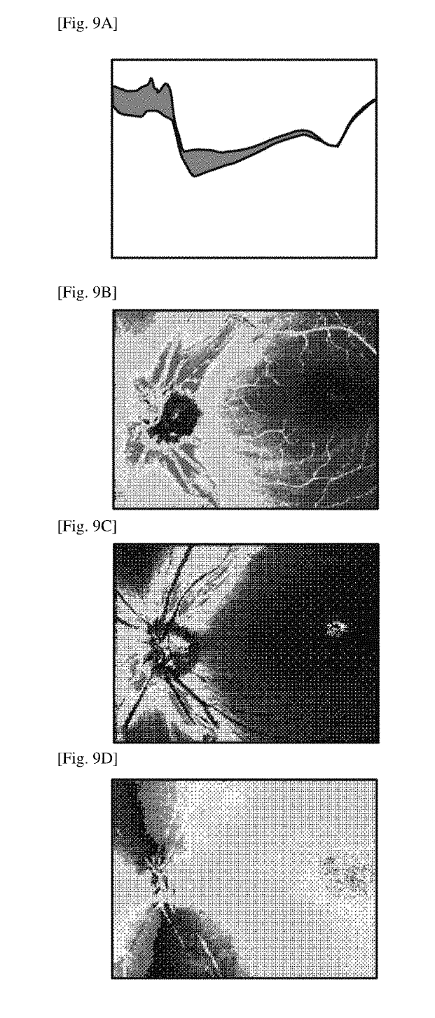

When a luminance image is generated, a map generation unit 148 as one function of the signal processing unit 144 performs segmentation by using the luminance image in step S804, thereby specifically extracting only a region corresponding to a nerve fiber layer, as shown in FIG. 9A. A method of specifically extracting a nerve fiber layer is the same as that described in the first embodiment, and hence a description of the method will be omitted. Although not described in this embodiment, it is possible to reduce the influences of blood vessels and the like in an image by additionally performing a general image processing method, for example, morphology processing.

(Generation of En Face Map)

Upon specifically extracting a nerve fiber layer, the map generation unit 148 generates a luminance image map, retardation map, orientation map, and nerve fiber layer thickness map in step S805.

(Generation of Luminance Image Map)

The map generation unit 148 generates a luminance image map. This luminance image map is generated by averaging luminance values in each A-scan direction, that is, each thickness direction, of the luminance image generated in step S803, and two-dimensionally arranging the average values, in the same manner as the generation of a luminance image map described in the first embodiment.

(Generation of Nerve Fiber Layer Thickness Map)

The map generation unit 148 generates a nerve fiber layer thickness map based on the extracted nerve fiber layer data. A nerve fiber layer thickness map two-dimensionally expresses the numbers of voxels in the A-scan direction counted for each pixel in a plane (x-y plane) perpendicular to the optical axis direction between the top and the bottom of nerve fiber layer in the nerve fiber layer volume data extracted in step S804. In this embodiment, averaging processing is performed by setting a window. A window of 6 pixels×2 pixels in the main scanning direction (x direction) and the sub-scanning direction (y direction) is provided for the luminance image information in the nerve fiber layer region acquired in step S804, and all the pixel counts included in the window are averaged to obtain a representative value. Sequentially shifting the window will acquire thickness information at all the pixels in the x-y plane. In FIG. 9B, FIG. 9B shows an example of a nerve fiber layer thickness map.

Note that in this embodiment, the window of 6 pixels×2 pixels is set in the x-y plane to perform the above processing. However, the present invention is not limited to this, and the examiner can arbitrarily set a window. In addition, it is possible to use other general averaging processing techniques.

(Generation of Retardation Map)

The map generation unit 148 generates a retardation map by using the retardation data of regions, of the plurality of B-scan retardation images generated in step S803, which correspond to the bottom of the nerve fiber layer. First of all, the map generation unit 148 applies the coordinate values of the nerve fiber layer region extracted in step S804 to each retardation image to extract a nerve fiber layer region from each retardation image. The respective retardation images are arranged in the y-scan direction to generate the volume data of the retardation image of the nerve fiber layer. A window in an x-y plane is then set for the volume data of the retardation image of the nerve fiber layer. The retardations of all the voxels in a region corresponding to the bottom of the nerve fiber layer included in the window are averaged to obtain a representative value in the window. In this embodiment, the window size is set to 6 pixels (x)×2 pixels (y). The window is sequentially shifted to finally display a retardation distribution as a two-dimensional image. In FIG. 9C, FIG. 9C shows an example of a retardation map.

Although in this embodiment, the window size is set to 6 pixels×2 pixels, the present invention is not limited to this, and the examiner can arbitrarily set a window size. In addition, it is possible to use other general averaging processing techniques.

Although retardation map is generated by using retardation data of regions which correspond to the bottom of the nerve fiber layer in this embodiment, the present invention is not limited to this. It is possible to use retardation data from layers located below the nerve fiber layer that preserve the polarization state for averaging, typically ranging from the bottom of the nerve fiber layer to the outer ploxiform layer. In addition, the examiner can arbitrarily set regions as needed. By doing in this way, it is possible to generate the retardation map with higher signal-to-noise ratio.

(Generation of Orientation Map)

The map generation unit 148 generates an orientation map in the nerve fiber layer region extracted in step S804. First of all, as also described in the first embodiment, the map generation unit 148 generates the volume data of orientation by obtaining a phase difference ΔΦ between tomographic signals A_{H} and A_{V} acquired in step S802 for each pixel of the volume data. The signal processing unit 144 then provides a window in an x-y plane to average orientation values of all the pixels existing in the window, and sets the obtained average value as a representative value of the window. The window is then sequentially shifted to obtain values with respect to all the pixels. The obtained values are two-dimensionally displayed to generate an orientation map. In this embodiment, a window of 6 pixels (x)×2 pixels (y) is used, and the values at all the pixels existing in the window are averaged to obtain a representative value. In FIG. 9D, FIG. 9D shows an example of an orientation map.

Although in this embodiment, the above processing is performed by using the window of 6 pixels×2 pixels, the present invention is not limited to this, and the examiner can arbitrarily set a window size. In addition, an averaging processing method to be used is not limited to this, and it is possible to use other general averaging processing techniques.

For example, averaging by methods in which retardation and axis orientation are averaged in a combined manner, such as by averaging the stokes vectors.

(Registration)

The map generation unit 148 then performs registration processing for a plurality of three-dimensional data in step S806. Registration is the processing of correcting positional shifts such as shifts and rotations between a plurality of images.

The map generation unit 148 uses five luminance image maps generated in step S805 to perform registration in an x-y plane. The map generation unit 148 uses one of the generated luminance image maps as a reference image, and calculates the cross-correlations between the reference image and the remaining four luminance images in the x-y plane to obtain correlation coefficients. The map generation unit 148 shifts and rotates the image so as to obtain the maximum correlation coefficient. Note that a reference image may be any one of the plurality of acquired luminance images. The examiner may arbitrarily select an image while avoiding any image determined to have low image quality, such as an image with many noise components. In addition, although in this embodiment, registration is performed by using a correlation coefficient, the present invention is not limited to this. It is possible to use other general registration techniques.

The map generation unit 148 then performs interpolation associated with the values of polarization parameters (retardation and orientation) for the four shifted and rotated luminance image maps. At this time, weights are provided in consideration of the following two points.

First, weights are provided based on the distances in the x and y directions between a pixel to be interpolated (target pixel) and four adjacent pixels. This is a technique generally called a bilinear method, which linearly decides the value of a target pixel in accordance with the distance between two points.

Second, weights are provided based on voxel counts for the calculation of polarization parameters for the four adjacent pixels. This is a method of considering voxel counts used for the calculation of a representative value of polarization parameters at each pixel in step S805. This makes the averaged contributions from voxels to a target pixel balanced regardless of voxel counts. Therefore, this improves the accuracy of polarization parameters at a target pixel.

According to the above method, letting σ1 be a polarization parameter at pixel A, N1 be the number of voxels contained in it, σ2 be a polarization parameter at pixel B, N2 be the number of voxels contained in it, and a:(1−a) be the ratio of distances from the target pixel, a polarization parameter a at the target pixel is expressed by equation (4).

$$σ = \frac{N 1 \times σ1 \times a + N 2 \times σ2 \times ( 1 - a )}{N 1 + N 2} \qquad ( 4 )$$

Executing the above processing on all luminance image maps makes it possible to perform registration in an x-y plane between a plurality of images.

Note that this embodiment is configured to perform interpolation by the method of applying weights based on voxel counts in addition to the bilinear method. However, the present invention is not limited to this. It is possible to use all other generally known interpolation methods.

(Volume Averaging)

When the map generation unit 148 performs registration in an x-y plane with respect to five sets of volume data in step S806, averaging processing is performed on polarization parameters and nerve fiber layer thicknesses with respect to the five sets of volume data. Averaging is executed between corresponding pixels, and weights are provided based on the numbers of voxels used for the generation of maps.

Although in this embodiment, averaging is performed by providing weights based on voxel counts, the present invention is not limited to this. It is possible to use other generally known averaging processing methods.

Performing the above processing makes it possible to generate a retardation map, orientation map, and nerve fiber layer thickness map by averaging five sets of volume data.

(Generation of Fiber Bundle Orientation Map)

The map generation unit 148 generates various types of maps by averaging processing in step S807, and then generates a fiber bundle orientation map in step S808. The map generation unit 148 generates a fiber bundle orientation map by using the nerve fiber layer thickness map generated in step S807. Although a fiber bundle orientation map is generated by using the nerve fiber layer thickness map in this embodiment, the present invention is not limited to this. It is possible to use other image like SLO intensity map.

First of all, the map generation unit 148 applies a high-pass filter to the nerve fiber layer thickness map generated in step S807. More specifically, the map generation unit 148 provides a window of 15 pixels×15 pixels for the nerve fiber layer thickness map, and subtracts the average thickness value in the window from the corresponding window region on the nerve fiber layer thickness map. With this operation, a region thicker or thinner than the average thickness in the window is highlighted. A nerve fiber layer thickness local change map is generated by performing this processing on the entire nerve fiber layer thickness map while shifting the window. Note that this nerve fiber layer thickness local change map mainly includes two elements. That is, they are thickness changes of a nerve fiber bundle and a blood vessel. Both a nerve fiber bundle and a blood vessel have tubular structures, but the blood vessel is larger in outer diameter than the nerve fiber bundle. For this reason, a threshold is provided for a nerve fiber layer thickness local change map to remove, from the local change map, a portion exhibiting a large thickness change as a blood vessel. As a result, the nerve fiber layer thickness local change map contains only thickness change information associated with the nerve fiber bundle. That is, thickness changes remaining on the nerve fiber layer thickness local change map grasp irregularity of the nerve fiber bundle, and a direction perpendicular to the irregularity indicates the running direction of the nerve fiber bundle.

Although this embodiment is configured to detect irregularity by setting a window of 15 pixels×15 pixels and subtracting the average thickness value in the window, the present invention is not limited to this. It is possible to use other generally known high-pass processing methods. In addition, the examiner can arbitrarily set a window size, as needed.

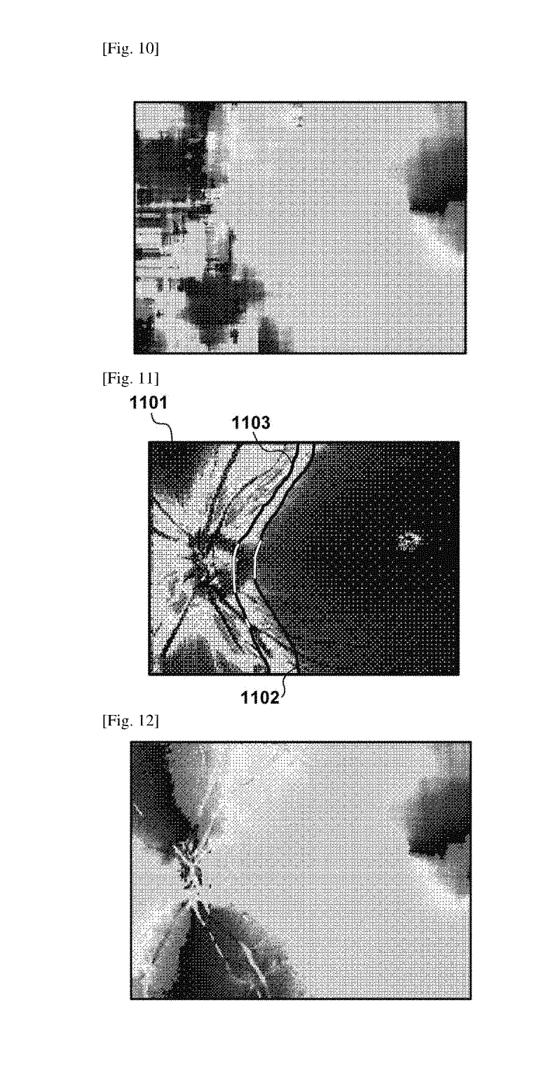

The map generation unit 148 then detects a thickness gradient direction at each pixel on the nerve fiber layer thickness local change map. That is, the map generation unit 148 detects the running direction of the nerve fiber bundle at each pixel. First of all, the map generation unit 148 applies a differential filter to the nerve fiber layer thickness local change map. This embodiment uses a Sobel filter as a differential filter. This obtains information about the magnitude and direction of an irregularity gradient at each pixel on the nerve fiber layer thickness local change map. An evaluation window is then set for each pixel. In this embodiment, an evaluation window of 120 pixels×120 pixels is set. A representative value in the thickness gradient direction at each pixel is then decided by the least squares estimation method. A fiber bundle orientation map can be generated by performing this processing on all the pixels on the nerve fiber layer thickness local change map. FIG. 10 shows an example of a fiber bundle orientation map.

Although in this embodiment, the thickness change gradient of a nerve fiber layer is calculated by using a Sobel filter, the present invention is not limited to this. In addition, the embodiment is configured to decide a thickness gradient direction at each pixel by setting an evaluation window of 120 pixels×120 pixels. However, the present invention is not limited to this. It is possible to use any pattern recognition techniques generally known as fingerprint authentication techniques.

(Generation of Fusion Map)

Upon generating an orientation map in step S805 and a fiber bundle orientation map in step S808, the map generation unit 148 generates a fusion map. This is because of the following reason. The orientation map generated in step S805 represents reliable orientation information at a portion around the optic papilla because the nerve fiber layer at the portion is sufficiently thick. However, at a portion around macula, the nerve fiber layer at the portion is thin, and hence the reliability of the orientation information is low. In contrast to this, the fiber bundle orientation map generated in step S808 represents reliable orientation information at a portion around macular because there are few thick blood vessels at the portion. However, at a portion near the optic papilla, there are many thick blood vessels, and hence data is missing. Therefore the data at this portion is not reliable. For this reason, it is possible to detect the running direction of nerve fiber bundles in a wide fundus region including the region from the optic papilla to the macula by using orientation map information for the portion around the optic papilla while using the fiber bundle orientation map information for the portion around the macula. Therefore, a fusion map is generated by combining an orientation map on the optic papilla side and a fiber bundle orientation map on the macula side.

A procedure for generating a fusion map by using a fiber bundle orientation map and an orientation map will be described with reference to FIG. 11.

First of all, the map generation unit 148 decides a position at which two images are to be combined. In this embodiment, the map generation unit 148 decides this position based on a retardation map 1101. The map generation unit 148 extracts a line 1102 having the first retardation and a line 1103 having the second retardation between the optic papilla and the macula. At this time, the first retardation and the second retardation have different values, and the examiner can arbitrarily set values. In the embodiment, the first retardation is 5°, and the second retardation is 7°.

The map generation unit 148 then arranges the fiber bundle orientation map so as to align it with a region on the macula side relative to the line 1102 having the first retardation, that is, on the right side of the line 1102 having the first retardation on a retardation map 1101. On the other hand, the map generation unit 148 arranges the orientation map so as to align it with a region on the optic papilla side relative to the line 1103 having the second retardation, that is, a region on the left side of the line 1103 having the second retardation on the retardation map 1101. The map generation unit 148 linearly interpolates the orientation map and the fiber bundle orientation map with each other and combines the two maps in the region sandwiched between the line 1102 having the first retardation and the line 1103 having the second retardation. FIG. 12 shows the fusion map generated in this manner

Although in this embodiment, a position at which two images are to be combined is decided based on retardation maps, the present invention is not limited to this. The examiner may decide a combining position in accordance with arbitrary maps, as needed. The examiner may arbitrarily execute this operation within the range in which nerve fiber bundle information is not lost. In addition, in the embodiment, in the region between an orientation map and a fiber bundle orientation map, the maps are combined with each other upon linear interpolation of the respective images. However, the present invention is not limited to this. It is possible to use any other generally known interpolation method.

(Nerve Fiber Bundle Tracing)

Upon generating a fusion map, the map generation unit 148 traces a nerve fiber bundle based on the fusion map. A method of tracing a nerve fiber bundle is the same as that described in the first embodiment, and hence a description of the method will be omitted.

Performing the above processing makes it possible to trace a nerve fiber bundle. In addition, although in this embodiment, a position at which an orientation map and a fiber bundle orientation map are to be jointed to each other is decided by using a retardation image, the present invention is not limited to this. It is possible to perform this operation by using a birefringence map or nerve fiber layer thickness map. In addition, although in the embodiment, the boundaries between an orientation map and a fiber bundle orientation map are a line having a retardation of 5° and a line having a retardation of 7, respectively, the present invention is not limited to this. The examiner can arbitrarily set boundaries, as needed. In addition, the embodiment includes the fundus image generation unit 147 and the map generation unit 148 each as one function of the signal processing unit 144. If, however, the signal processing unit 144 does not have such distinctive functions and processes, the signal processing unit 144 may be configured to generate fundus images and maps by itself.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

### DESCRIPTION OF REFERENCE NUMERALS

143 control unit
144 signal processing unit
145 drive control unit
146 display unit
147 fundus image generation unit
148 map generation unit
149 display control unit

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-017908, filed Jan. 30, 2015 which is hereby incorporated by reference herein in its entirety.

1. A tomographic imaging apparatus comprising:
a generation unit configured to generate a nerve fiber bundle map;
a designation unit configured to designate an arbitrary nerve fiber bundle in the nerve fiber bundle map; and
a display control unit configured to cause a display unit to display a parameter of the designated nerve fiber bundle.

2. The tomographic imaging apparatus according to claim 1, wherein the display control unit highlights the designated nerve fiber bundle displayed by the display unit.

3. The tomographic imaging apparatus according to claim 1, wherein the parameter includes one of luminance information, retardation information, orientation information, birefringence information, and layer thickness information.

4. The tomographic imaging apparatus according to claim 1, further comprising a fundus image generation unit configured to generate a luminance image and a polarization characteristic image of a fundus of an eye to be examined,
wherein the generation unit generates the nerve fiber bundle map based on the luminance image and the polarization characteristic image.

5. A tomographic imaging apparatus comprising:
a fundus image generation unit configured to generate a luminance image and a polarization characteristic image of a fundus of an eye to be examined, and
a generation unit configured to generate a nerve fiber bundle map based on the luminance image and the polarization characteristic image.

6. The tomographic imaging apparatus according to claim 4, wherein the polarization characteristic image comprises an image based on retardation and an image based on orientation, and
wherein the generation unit generates, on the basis of the image based on retardation, the nerve fiber bundle map by combining an image based on the luminance image and the image based on orientation.

7. The tomographic imaging apparatus according to claim 4, wherein the polarization characteristic image comprises an image based on retardation and an image based on orientation, and
wherein the generation unit decides, on the basis of the image based on retardation, a position at which the luminance image and the image based on orientation are to be combined, and the generation unit generates, on the basis of the decided position, the nerve fiber bundle map by combining an image based on the luminance image and the image based on orientation.

8. The tomographic imaging apparatus according to claim 1, further comprising a tracing unit configured to trace a nerve fiber bundle based on the nerve fiber bundle map.

9. The tomographic imaging apparatus according to claim 1, further comprising:
a fundus image generation unit configured to generate a luminance image and a polarization characteristic image of a fundus of an eye to be examined; and
a tracing unit configured to trace a nerve fiber bundle based on the luminance image and the polarization characteristic image.

10. The tomographic imaging apparatus according to claim 8, wherein the display control unit is configured to cause a display unit to display a nerve fiber bundle trace map based on the traced nerve fiber bundle.

11. A tomographic imaging apparatus comprising:
a fundus image generation unit configured to generate a luminance image and a polarization characteristic image of a fundus of an eye to be examined; and
a tracing unit configured to trace a nerve fiber bundle based on the luminance image and the polarization characteristic image.

12. The tomographic imaging apparatus according to claim 11, further comprising a display control unit configured to cause a display unit to display a nerve fiber bundle trace map based on the traced nerve fiber bundle.

13. A tomographic imaging method comprising:
generating a nerve fiber bundle map;
designating an arbitrary nerve fiber bundle in the nerve fiber bundle map; and
causing a display unit to display a parameter of the designated nerve fiber bundle.

14. A tomographic imaging method comprising:
generating a luminance image and a polarization characteristic image of a fundus of an eye to be examined; and
generating a nerve fiber bundle map based on the luminance image and the polarization characteristic image.

15. A tomographic imaging method comprising:
generating a luminance image and a polarization characteristic image of a fundus of an eye to be examined; and
tracing a nerve fiber bundle based on the luminance image and the polarization characteristic image.

16. A non-transitory computer-readable storage medium storing a program for causing a computer to execute steps in the tomographic imaging method defined in claim 13.

17. An image processing apparatus comprising:
a first generation unit configured to generate a three-dimensional luminance image of a fundus of an eye to be examined, based on tomographic signals of different polarized light components obtained by dividing interference light;
an extraction unit configured to extract a nerve fiber layer of the three-dimensional luminance image;
a second generation unit configured to generate a map indicating a local change of the extracted nerve fiber layer and a map indicating a phase difference between the tomographic signals of different polarized light components; and
a third generation unit configured to generate a map indicating a running direction of a nerve fiber bundle of the fundus by combining a map indicating the local change with respect to a region including a macula of the fundus and a map indicating the phase difference with respect to a region including an optic papilla of the fundus.

18. The image processing apparatus according to claim 17, further comprising:
a decision unit configured to decide, as the region including the macula, a region in which a thickness of the nerve fiber layer is less than a threshold, and to decide, as the region including the optic papilla, a region in which the thickness of the nerve fiber layer is equal to or more than the threshold.

19. An image processing method comprising:
generating a three-dimensional luminance image of a fundus of an eye to be examined, based on tomographic signals of different polarized light components obtained by dividing interference light;
extracting a nerve fiber layer of the three-dimensional luminance image;
generating a map indicating a local change of the extracted nerve fiber layer and a map indicating a phase difference between the tomographic signals of different polarized light components; and
generating a map indicating a running direction of a nerve fiber bundle of the fundus by combining a map indicating the local change with respect to a region including a macula of the fundus and a map indicating the phase difference with respect to a region including an optic papilla of the fundus.

20. The image processing method according to claim 19, further comprising:
deciding, as the region including the macula, a region in which a thickness of the nerve fiber layer is less than a threshold, and deciding, as the region including the optic papilla, a region in which the thickness of the nerve fiber layer is equal to or more than the threshold.
